(19) United States
(12) Patent Application Publication
(10) Pub. No.: US 2007/0129582 A1
(43) Pub. Date: Jun. 7, 2007

(54) Production processes and systems

(76) Inventor: Mitchel Cohn, West Haven, CT (US)

Correspondence Address: WELLS ST. JOHN P.S., 601 W. FIRST AVENUE, SUITE 1300, SPOKANE, WA, 99201, US

(21) Appl. No.: 11/699,682

(22) Filed: Jan. 29, 2007

Related U.S. Application Data
(62) Division of application No. 11/271,531, filed on Nov. 10, 2005.

(51) Int. Cl. C07C 17/38

(52) U.S. Cl. 570/177

(57) ABSTRACT

Halocarbon production processes are described that can include providing a reactant mixture including a halogenating reagent and a halocarbon reactant. The processes can also include reacting the halogenating reagent and the halocarbon reactant to form a product mixture that can include a halocarbon product and at least a portion of the halogenating reagent, and phase separating the halocarbon product from at least a portion of the halogenating reagent. Systems for separating components of a mixture are described that can include a vessel configured to receive a mixture and maintain a temperature of the mixture within the vessel below about 7° C. The mixture can include a halogen exchange reactant and a halocarbon product. Separation processes are described that can include providing a mixture of a halogen exchange reactant and a halocarbon product. The process can include phase separating the mixture into a reactant phase and a product phase.

### CROSS REFERENCE TO RELATED APPLICATION

This is a Divisional of application Ser. No. 11/271,531, filed Nov. 10, 2005, the disclosure of which is incorporated by reference herein.

### TECHNICAL FIELD

Production processes and systems are described. More particularly halocarbon production processes and systems are described that may be utilized to produce compounds such as hexafluoropropane (C₃F₆H₂, HFC-236).

### BACKGROUND OF THE INVENTION

Production processes and systems are utilized to produce compounds such as halocarbon compounds, including compounds such as hydrofluorocarbons such as hexafluoropropane. Halocarbon compounds such as hexafluoropropane have many uses. For example, these compounds can be used as fire extinguishants, refrigerants, foam blowing agents, and/or propellants.

Manufacturing halocarbon compounds is not trivial as the processes can utilize compounds such as HF, KF, F₂, and/or NF₃, all of which having stringent handling guidelines to ensure the safety of the process. As has been documented, these processes can produce azeotropic and/or azeotrope-like mixtures of the product compound, hexafluoropropane for example, and reactants, HF for example, making the separation of the product compounds from the reactants difficult. The present disclosure provides processes and systems that can be utilized to produce halocarbon compounds.

### SUMMARY OF THE INVENTION

Halocarbon production processes are described that can include providing a reactant mixture including a halogenating reagent and a halocarbon reactant. Moles of the halogenating reagent within the reactant mixture can exceed moles of the halocarbon reactant, according to exemplary embodiments. The processes can also include reacting the halogenating reagent and the halocarbon reactant to form a product mixture that can include a halocarbon product and at least a portion of the halogenating reagent. Phase separating can be employed to separate the halocarbon product from at least a portion of the halogenating reagent.

Systems for separating components of a mixture are described that can include a vessel configured to receive a mixture and maintain a temperature of the mixture within the vessel below about 7° C. The mixture can include a halogen exchange reactant and a halocarbon product.

Separation processes are described that can include providing a mixture that can include a halogen exchange reactant and a halocarbon product. The process can include phase separating the mixture into a reactant phase and a product phase.

### BRIEF DESCRIPTION OF THE FIGURES

FIG. 1 is a production system according to an embodiment.

FIG. 2 is the production system of FIG. 1 according to an embodiment.

### DETAILED DESCRIPTION OF THE PREFERRED EMBODIMENTS

This disclosure is submitted in furtherance of the constitutional purposes of the U.S. Patent Laws “to promote the progress of science and useful arts” (Article 1, Section 8).

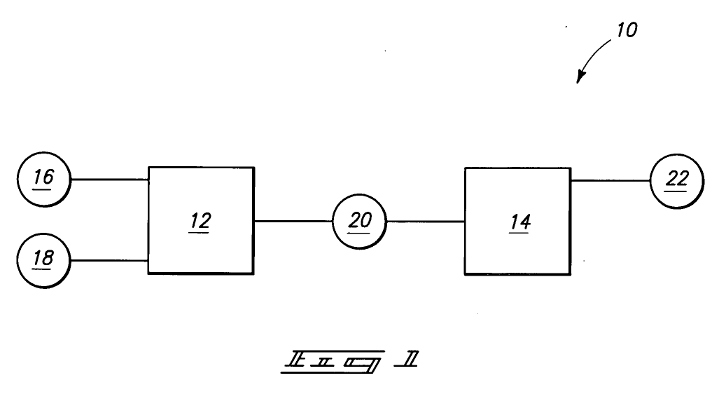

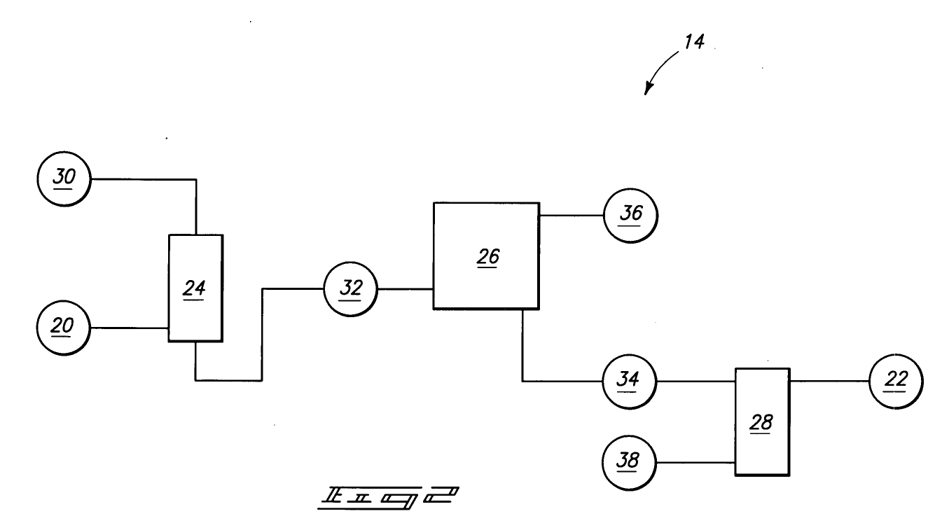

Exemplary methods and systems are described with reference to FIGS. 1 and 2. Referring to FIG. 1, a halocarbon production system 10 is shown that includes at least two zones, a reaction zone 12 and a separation zone 14. Reaction zone 12 can be configured to receive reactants such as reactants 16 and 18 and prepare a product mixture 20 which can be separated into a product 22 in separation zone 14.

Reactant 16 can include a halogenating reagent such as a halogen exchange reactant that may include one or more of hydrogen, fluorine, chlorine, bromine, and/or iodine. Reactant 16 can include both hydrogen and fluorine, in exemplary embodiments, and in other embodiments reactant 16 can include HF in fresh or recycled form. Reactant 16 can be stored in a vessel (not shown) an provided to zone 12 using pressure differential devices including but not limited to pressure differential pumps and/or flow meters, for example. As halogenating reagents may be corrosive, devices for storing and/or transferring reactant 16 can be configured to resist such corrosion.

Reactant 18 can include a halocarbon reactant such as a C-3 compound including, but not limited to, C-3 compounds that include fluorine. Reactant 18 can also include a heterohalogenated compound such as C-3 compounds that include both fluorine and chlorine, for example. In exemplary embodiments, reactant 18 can include hexachloropropane (C₃Cl₆H₂). In exemplary embodiments, reactant 18 can also be referred to as a halogen. Reactant 18 can be combined with reactant 16 either prior to entering zone 12 or within zone 12. Reactant 18, such as hexachloropropane can be purchased and/or manufactured according to accepted industry methods. Exemplary methods and systems for manufacturing hexachloropropane, for example, include those described in U.S. patent application Ser. No. 10/916,275 entitled “Catalyst Preparation Processes, Catalyst Regeneration Processes, and Halocarbon Production Processes” filed Aug. 10, 2004, the entirety of which is incorporated by reference herein.

Upon mixing of reactant 16 and 18 a reaction mixture can be formed within reaction zone 12. Reactant 16 and 18 can be provided to reaction zone 12 in particular mole ratios. According to exemplary embodiments the mole ratio of reactant 16 to reactant 18 can be at least 6:1 and according to other embodiments the mole ratio of reactant 16 to reactant 18 can be greater than 6:1 thereby establishing a reaction mixture that is rich in reactant 16. The mole ratio of reactant 16 to reactant 18 can be from about 8 to about 12, for example. In exemplary embodiments, reactant 16 and 18 can be provided to reaction zone 12 using flow meters. For example, the flow rates of the flow meters providing reactant 16 and reactant 18 to reaction zone 12 can be configured to provide reactant 16 at a higher rate to reaction zone 12 than the flow rate of reactant 18 is provided to reaction zone 12, for example.

Reaction zone 12 can be configured as a single reactor or multiple reactors to receive both reactants 16 and 18 separately or a mixture of reactants 16 and 18. Exemplary reactors can include reactors configured as a liquid phase reactors. Liquid phase reactors can be configured to react at least one composition while the composition is in its liquid form. All reactants within the reactor may be in the liquid form, for example, or at least one of the reactants may be in liquid form. According to exemplary embodiments, reactants can be maintained as a liquid through adjustment of the temperatures and/or pressures of the contents of the reactor.

Within a reactor of reaction zone 12 configured to receive reactant 16 and 18, a catalyst, such as a liquid phase catalyst, may be provided. Exemplary liquid phase catalysts include catalyst compositions containing Sb, such as SbCl₅. Exemplary catalysts and catalyst preparations are described in U.S. patent application Ser. No. 10/916,275 entitled “Catalyst Preparation Processes, Catalyst Regeneration Processes, and Halocarbon Production Processes” filed Aug. 10, 2004, the entirety of which is herein incorporated by reference. Reactants 16 and 18 can be combined to form a reaction mixture and the reaction mixture can be exposed to the liquid phase catalyst, for example, within zone 12.

Upon formation of the reaction mixture including reactant 16 and 18, a product mixture 20 can be formed. Product mixture 20 can include a halocarbon product and at least a portion of the reactant 16, such as halogenating reagent, for example. According to exemplary embodiments the halocarbon product can include fluorine. The product can also include both hydrogen and fluorine. In exemplary embodiments, the halogenated product can be saturated and, in other embodiments, the halogenated product can be a saturated C-3 compound. The halocarbon product can also include C₃F₆H₂, such as CF₃CH₂CF₃.

In a particular embodiment, for example and by way of example only, reactant 16 can include HF, reactant 18 can include C₃Cl₆H₂ and the product mixture 20 can be produced through a halogen exchange reaction of HF and C₃Cl₆H₂ to produce the halocarbon product C₃F₆H₂. Product mixture 20 can be transferred into separation zone 14 to produce a product 22.

Referring to FIG. 2, an exemplary separation zone 14 is depicted that can include separation apparatus 24, 26, and 28. As exemplarily depicted the separation apparatus can be configured to distill mixture 20 in apparatus 24, phase separate the resulting product of distillation from apparatus 24 in apparatus 26, and then scrub the resulting product of phase separation in 26 within apparatus 28. As exemplarily depicted in FIG. 2, zone 14 is for example purposes only and should not be used to limit the scope of the claimed invention for at least the reason that many configurations of zone 14 are contemplated.

As referred to earlier, mixture 20 can include a halogen exchange reactant and a halocarbon product. Mixture 20 can also include additional by-products from reaction zone 12 such as HCl, for example. Apparatus 24 can be configured as a distillation apparatus and product mixture 20 can be transferred to apparatus 24 and by-products 30, such as HCl can be separated from mixture 20 to form a subsequent mixture 32. Mixture 32 can include a halogen exchange reactant and a halocarbon product, for example.

Mixture 32 can be transferred to apparatus 26 which can be configured as a phase separation apparatus. Apparatus 26 can be configured as a vessel that is coupled to reaction zone 12. Such coupling can include coupling via apparatus 24 and/or apparatus 26 can be coupled directly to reaction zone 12. For example, and by way of example only, the reactant such as HF and C₃Cl₆H₂ can be combined to form a mixture that comprises HF, C₃F₆H₂ and HCl. This mixture can be transferred to apparatus 24 wherein HCl is removed as by-product 30 and the remaining HF and C₃F₆H₂ is transferred to apparatus 26 for phase separation.

Upon transfer of mixture 32 to apparatus 26, the temperature of mixture 32 can be lowered to form at last two phases within apparatus 26. In exemplary embodiments the phases can include a reactant phase and a product phase. The reactant phase can include the halogen exchange reactant such as HF. The product phase can include the halocarbon product such as C₃F₆H₂, for example. According to exemplary embodiments the temperature of mixture 32 within apparatus 26 can be reduced to below about 7° C. and in other exemplary embodiments the temperature can be reduced to above −30° C. The temperature of mixture 32 within apparatus 26 can be from about −30° C. to about 7° C., for example.

Apparatus 26 can be configured to separate the product phase from the reactant phase, for example. In exemplary embodiments apparatus 26 can also be configured to remove either one or both of the phases from the vessel. Separating the product phase from the reactant phase using apparatus 26 can include removing either one or both of the phases from the vessel such as separating reactant mixture 36 from product mixture 34. As such, apparatus 26 can be configured to remove the product phase within the vessel selective to the reactant phase within the vessel. For example, apparatus 26 can be configured with a stand pipe having an opening configured to remove the upper phase selective to the lower phase. Apparatus 26 can be cooled to temperatures as low as −30° C. by configuring apparatus 26 as a vessel equipped with a cooling jacket having a glycol or brine solution. For example, product mixture 34 can be removed from a lower portion of apparatus 26 while a reactant mixture 36 can be removed from an upper portion of apparatus 26. Product mixture 34 can include the product phase and/or contain a product such as C₃F₆H₂ and less than about 2% (wt./wt.) of halogen exchange reactant, for example HF. Reactant mixture 36 may contain the reactant phase and may be returned or recycled to another portion of system 10 including, but not limited to, reaction zone 12. Apparatus 26 can be configured to continuously separate reactant mixture 36 from product mixture 34 as mixture 32 is received from zone 12.

For example and by way of example only, about 301 lb/hr of mixture containing about a 3.36 mole ratio of HF to HFC-236fa can be fed into a 3/16 inch stainless steel jacketed tank having an internal temperature of −20C and a pressure of 1 atm (=101.325 Kpa=1.10325 bar=1.033 Kg/cm²). Upper and lower phases can be formed and the upper layer can be removed at about 136.4 lb/hr containing about 65.7% (wt./wt.) HF or about a 14.6 molar ratio of HF to HFC-236fa. The lower phase can be removed at about 164.4 lb/hr containing about 1.6% (wt./wt.) HF or about a 0.12 molar ratio of HF to HFC-236fa.

As shown in FIG. 2, apparatus 26 is coupled to apparatus 28. Product mixture 34 may be conveyed to apparatus 28 for what can be referred to as scrubbing. In exemplary embodiments, scrubbing can include washing of the product with an aqueous solution and the subsequent drying of the washed product to remove residual water. For example, product mixture 34 can be combined with an aqueous solution. The aqueous solution can contain Na and/or K, and/or have a pH greater than 7, such as a basic solution, for example. Particularly, upon combining the aqueous solution with product mixture 34 within apparatus 28, organic-aqueous phase separation can be performed between a primarily organic halocarbon product such as C₃F₆H₂ and an aqueous solution which can contain the majority of HF that remained in product mixture 34. The phase separation can separate halocarbon product 22 from aqueous solution 38. Halocarbon product 22 can further be purified via drying techniques using molecular sieve for example and/or high purity distillation techniques known to those of ordinary skill in the art.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

1. A separation process comprising:
providing a mixture comprising a halogen exchange reactant and a halocarbon product; and
phase separating the mixture into a reactant phase and a product phase.

2. The process of claim 1 wherein the mixture is produced during a halogen exchange reaction utilizing the halogen exchange reactant.

3. The process of claim 1 wherein the halogen exchange reactant comprises fluorine.

4. The process of claim 3 wherein the halogen exchange reactant comprises hydrogen and fluorine.

5. The process of claim 1 wherein the halogen exchange reactant is HF.

6. The process of claim 1 wherein the halocarbon product is a C-3 compound.

7. The process of claim 1 wherein the halocarbon product comprises fluorine.

8. The process of claim 1 wherein the halocarbon product comprises both hydrogen and fluorine.

9. The process of claim 1 wherein the halocarbon product is saturated.

10. The process of claim 1 wherein the halocarbon product is a saturated C-3 compound.

11. The process of claim 1 wherein the halocarbon product is C₃F₆H₂.

12. The process of claim 1 wherein the halocarbon product is CF₃CH₂CF₃.

13. The process of claim 1 wherein the phase separating the mixture comprises:
transferring the mixture to within a vessel; and
while the mixture is within the vessel, lowering the temperature of at least a portion of the mixture within the vessel to form both the reactant phase and the product phase.

14. The process claim 13 wherein the lowering the temperature of at least a portion of the mixture comprises lowering the temperature of the portion to a temperature below about 7° C.

15. The process of claim 13 further comprising separating the product phase from the reactant phase.

16. The process of claim 15 wherein the separating the product phase from the reactant phase comprises removing either one or both of the phases from the vessel.

17. The process of claim 13 wherein the vessel is configured to remove the product phase within the vessel selective to the reactant phase within the vessel.

18. The process of claim 17 wherein the product phase comprises the halocarbon product.

19. The process of claim 17 wherein the reactant phase comprises the halogen exchange reactant.

20-28. (canceled)

29. A halocarbon production process comprising:
providing a reactant mixture comprising a halogenating reagent and a halocarbon reactant, wherein moles of the halogenating reagent exceeds moles of the halocarbon reactant within the reactant mixture;
reacting the halogenating reagent and the halocarbon reactant to form a product mixture comprising a halocarbon product and at least a portion of the halogenating reagent; and
phase separating the halocarbon product from the halogenating reagent.

30. The process of claim 29 wherein the providing the reactant mixture comprises transferring the halogenating reagent to a reactor at a first flow rate and transferring the halocarbon reactant to the reactor at a second flow rate, wherein the first flow rate is greater than the second flow rate.

31. The process of claim 30 wherein the mole ratio of the halogenating reagent to the halocarbon reactant is greater than about 6.

32. The process of claim 30 wherein the mole ratio of the halogenating reagent to the halocarbon reactant is from about 8 to about 12.

33. The process of claim 31 wherein:
the halogenating reagent is HF; and
the halocarbon reactant is C₃Cl₆H₂.

34. The process of claim 29 wherein the reacting the halogenating reagent and the halocarbon reactant comprises exposing the halogenating reagent to the halocarbon reactant in the presence of a liquid phase catalyst.

35. The process of claim 34 wherein the liquid phase catalyst comprises Sb.

36. The process of claim 35 wherein the liquid phase catalyst further comprises Cl.

37. The process of claim 34 wherein:
the halogenating reagent is HF;
the halocarbon reactant is C₃Cl₆H₂; and
the liquid phase catalyst comprises SbCl₅.

38. The process of claim 29 wherein the phase separating comprises:
transferring the product mixture to within a vessel; and
while the product mixture is within the vessel, lowering the temperature of at least a portion of the product mixture within the vessel to form both a reactant phase and a product phase.

39. The process claim 38 wherein the lowering the temperature of at least a portion of the product mixture comprises lowering the temperature of the portion to a temperature below about 7° C.

40. The process of claim 38 wherein the phase separating further comprises removing either one or both of the phases from the vessel.

41. The process of claim 38 wherein the vessel is configured to remove the product phase within the vessel selective to the reactant phase within the vessel.

42. The process of claim 38 wherein the product phase comprises the halocarbon product.

43. The process of claim 38 wherein the reactant phase comprises the portion of the halogenating reagent.

44. The process of claim 38 wherein:
the halocarbon product is C₃F₆H₂; and
the halogenating reagent is HF.
